(19) United States
(12) Patent Application Publication
(10) Pub. No.: US 2021/0236521 A1
(43) Pub. Date: Aug. 5, 2021

(54) Method For Improving Brain Function And A Dietary Supplement For Use In The Method

(71) Applicant: RB Health (US) LLC, Parsippany, NJ (US)

(72) Inventors: Alejandra Araya Gratson, Parsippany, NJ (US); Jane Mary Barracato, Parsippany, NJ (US); Michal Jan Siwek, Salt Lake City, UT (US); Yegor Dmitrievich Chekmarev, Salt Lake City, UT (US); Kenneth Gene Rayl, Salt Lake City, UT (US)

(21) Appl. No.: 16/778,372

(22) Filed: Jan. 31, 2020

(51) Int. Cl. A61K 31/661; A61K 36/74; A61K 31/4415; A61K 31/714; A61K 31/522; A23L 33/105; A23L 33/15; A23L 33/12

(52) U.S. Cl. CPC A61K 31/661; A61K 36/74; A61K 31/4415; A61K 31/714; A61K 9/0053; A23L 33/105; A23L 33/15; A23L 33/12; A61K 31/522

(57) ABSTRACT

The present disclosure relates generally to a method of promoting brain function in a target subject by the administration of a dietary supplement composition. Additionally, the disclosure relates to a dietary supplement composition which promotes brain function. In particular, the dietary supplement composition comprises phosphatidylserine and coffee fruit extract.

### TECHNICAL FIELD

The present disclosure relates generally to a method of promoting brain function in a target subject by the administration of a dietary supplement composition. Additionally, the disclosure relates to a dietary supplement composition which promotes brain function. In particular, the dietary supplement composition comprises phosphatidylserine and coffee fruit extract.

### BACKGROUND

A dietary supplement is a product which is designed to be consumed by a human or animal to supplement the diet to provide nutrients in order to increase the intake of one or more nutrients. Often, such supplements contain vitamins and minerals which have been found to be essential to life. However, dietary supplements may also contain ingredients which have a beneficial biological effect, but which are not necessarily deemed essential to life.

Phosphatidylserine is a type of phospholipid found in cell membranes and particularly in the cells of the brain. It is vital for cognitive function as it is key to supporting cell function in the brain where it impacts the brain's structure and signal system. The intake of phosphatidylserine from food alone has declined over recent years. Studies have revealed phosphatidylserine contributes to cognitive function in individuals of various age groups. It is believed that young people can synthesize enough phosphatidylserine to maintain healthy brain function. However, during aging the phosphatidylserine content in the brain diminishes leading to neuronal chemical changes. Dietary supplements for cognitive impairment published in Acts Esp Psiquiatr 2017; 45 (Suppl. 1):37-47 states that “cerebral aging is associated with changes in the lipid composition of neuronal membranes” so it has been suggested that the treatment with phospholipids, like phosphatidylcholine and phosphatidylserine, could favour cognitive improvement.

Coffee fruit is the skin and rind which surrounds a coffee bean. It has been proven to elevate the body's level of the neuroprotein brain-derived neurotrophic factor (BDNF). BDNF is a vital neuroprotein that is known to strengthen connections between brain cells and helps establish new connections. It is also important for neuronal survival and growth, serves as a neurotransmitter modulator and participates in neuronal plasticity which is essential for learning and memory. It has been demonstrated in the British Journal of Nutrition, Vol. 110, Issue 3 that the consumption of whole coffee fruit can support the production of BDNF in a human.

The foregoing discussion is presented solely to provide a better understanding of the nature of the problems confronting the art and should not be construed in any way as an admission to prior art.

The present disclosure is directed, in an embodiment, to provide a method for improving brain function. In another embodiment, it is directed to a dietary supplement composition which improves brain function.

### BRIEF SUMMARY

In accordance with the foregoing objectives and others, the present disclosure provides a method for promoting brain function in a target subject. In certain embodiments, the target subject is a mammal including humans and companion animals. In some embodiments, the target subject is an adult human. In another embodiment of the invention, the present disclosure is directed to a composition suitable for use as a dietary supplement by combining phosphatidylserine and coffee fruit extract in a single composition. In some embodiments, it has surprisingly been found that a dietary supplement composition comprising coffee fruit extract and phosphatidylserine has a beneficial effect which is greater than the effect expected from phosphatidylserine and coffee fruit extract when administered individually.

In one aspect, the present disclosure is directed to a method of improving brain function in a target subject, the method comprising administering a dietary supplement composition comprising phosphatidylserine; and coffee fruit extract to the target subject. In some embodiments, the phosphatidylserine can be in an amount of about 75 mg to about 400 mg, preferably about 90 mg to about 300 mg and more preferably about 100 to about 200 mg. In some embodiments, the coffee fruit extract can be in an amount of about 80 mg to 300 mg and more preferably about 100 mg to 240 mg. In some embodiments, the dietary supplement composition can further comprise one or more vitamins. In some embodiments, the vitamins can comprise at least one of vitamin B6 and vitamin B12. In some embodiments, the vitamin B6 can be provided by one or more of pyridoxine, pyridoxal, pyridoxamine and pyridoxine hydrochloride. In some embodiments, the vitamin B6 can be present in an amount of about 1 mg to 4 mg, more preferably about 1.5 mg to about 2.5 mg. In some embodiments, the vitamin B12 can be provided by one or more of cobalamin, methylcobalamin, deoxyadenosylcobalamin, cyanocobalamin, hydroxocobalamin and adenosylcobalamin. In some embodiments, the vitamin B12 can be present in an amount of about 1 μg to about 11 μg, preferably about 2 μg to about 8 μg and more preferably about 2.4 μg to about 4.8 μg. In some embodiments, the dietary supplement composition can further comprise folate. In some embodiments, the folate can be provided by at least one of folic acid, folacin and pteroylglutamic acid. In some embodiments, the folic acid can be present in an amount of about 0.2 to about 1.0 mg, preferably about 0.4 mg to about 0.8 mg. In some embodiments, the dietary supplement composition can further comprise one or more ingredients selected from the group consisting of solvents, carriers, flow agents, antioxidants, excipients, lubricants, processing aids, colorants, water, and combinations thereof. In some embodiments, the dietary supplement composition is in a form selected from the group consisting of a solution, a capsule, a tablet, a gummy, a chewable, a melt in the mouth form, a liquid or a caplet. In some embodiments, the target subject is an adult human. In some embodiments, the coffee fruit extract has been decaffeinated.

In a related aspect, the present disclosure provides a dietary supplement composition comprising phosphatidylserine; and coffee fruit extract. In some embodiments, the phosphatidylserine can be in an amount of about 75 mg to about 400 mg, preferably about 90 mg to about 300 mg and more preferably about 100 to about 200 mg. In some embodiments, the coffee fruit extract can be in an amount of about 80 mg to 300 mg and more preferably about 100 mg to 240 mg. In some embodiments, the dietary supplement composition can further comprise one or more vitamins. In some embodiments, the vitamins can comprise at least one of vitamin B6 and vitamin B12. In some embodiments, the vitamin B6 can be provided by one or more of pyridoxine, pyridoxal, pyridoxamine and pyridoxine hydrochloride. In some embodiments, the vitamin B6 can be present in an amount of about 1 mg to 4 mg, more preferably about 1.5 mg to about 2.5 mg. In some embodiments, the vitamin B12 can be provided by one or more of cobalamin, methylcobalamin, deoxyadenosylcobalamin, cyanocobalamin, hydroxocobalamin and adenosylcobalamin. In some embodiments, the vitamin B12 can be present in an amount of about 1 μg to about 11 μg, preferably about 2 μg to about 8 μg and more preferably about 2.4 μg to about 4.8 μg. In some embodiments, the dietary supplement composition can further comprise folate. In some embodiments, the folate can be provided by at least one of folic acid, folacin and pteroylglutamic acid. In some embodiments, the folic acid can be present in an amount of about 0.2 to about 1.0 mg, preferably about 0.4 mg to about 0.8 mg. In some embodiments, the dietary supplement composition can further comprise one or more ingredients selected from the group consisting of solvents, carriers, flow agents, antioxidants, excipients, lubricants, processing aids, colorants, water, and combinations thereof. In some embodiments, the dietary supplement composition is in a form selected from the group consisting of a solution, a capsule, a tablet, a gummy, a chewable, a melt in the mouth form, a liquid or a caplet. In some embodiments, the coffee fruit extract has been decaffeinated.

In a related aspect, the present disclosure provides a dosage regimen for administering a dietary supplement composition to a target subject, the dietary supplement composition comprising phosphatidylserine; and coffee fruit extract, wherein the dosage regimen can comprise a daily dosage amount of the phosphatidylserine and the coffee fruit extract, wherein the dosage regimen can be optionally delivered in one or more unit doses.

These and other objects, features and advantages of the disclosure will become more apparent upon reading the following specification in conjunction with the accompanying description and claims.

### DETAILED DESCRIPTION OF THE DISCLOSURE

Reference will be made in detail to the embodiments of the present disclosure, one or more examples of which are set forth hereinbelow. Each example is provided by way of explanation of the dietary supplement composition of the present disclosure and is not a limitation. In fact, it will be apparent to those skilled in the art that various modifications and variations can be made to the teachings of the present disclosure without departing from the scope of the disclosure. For instance, features, illustrated or described as part of one embodiment can be used with another embodiment to yield a still further embodiment.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only and is not intended as limiting the broader aspects of the present disclosure.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

### Definitions

As used herein, all terms are intended to have their ordinary meaning in the art unless specifically defined.

“Adult” means a subject in age from 18 years upwards.

“Modulate” or “modulating” means exerting a modifying, controlling and/or regulating influence. In some embodiments, the term “modulating” means exhibiting an increasing or stimulatory effect on the level/amount of a particular component. In other embodiments, “modulating” means exhibiting a decreasing or inhibitory effect on the level/amount of a particular component.

All percentages, parts and ratios as used herein are by weight of the total formulation, unless otherwise specified.

The present disclosure relates generally to a method for improving brain function via administration of a dietary supplement composition comprising phosphatidylserine and coffee fruit extract. Additionally, the disclosure relates to a dietary supplement composition comprising phosphatidylserine and coffee fruit extract which is suitable for administration to a human subject.

The dietary supplement composition of the present disclosure may be substantially free of any optional or selected ingredients described herein, provided that the remaining nutritional composition still contains all of the required ingredients or features described herein.

All references to singular characteristics or limitations of the present disclosure shall include the corresponding plural characteristic or limitation, and vice versa, unless otherwise specified or clearly implied to the contrary by the context in which the reference is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

The present disclosure is directed to a method of improving brain function by the administration of a dietary supplement composition comprising phosphatidylserine and coffee fruit extract and to the composition itself. The administration of the dietary supplement composition results in improved focus, memory, learning, accuracy and concentration in a target subject. In certain embodiments, the combination of phosphatidylserine and coffee fruit extract is believed to have additive and/or synergistic beneficial effect over the expected effect from the administration of one of phosphatidylserine or coffee fruit extract. In some embodiments, as a result of the improved brain function other aspects such as a target subject's behavior may be modulated.

The method of improving brain function in a target subject is achieved by the administration of a dietary supplement composition comprising phosphatidylserine and coffee fruit extract via the gut-brain axis. Thus, in some embodiments, the present disclosure is directed to a method of modulating gut-brain communication, the method comprising administering to a target subject a nutritional composition comprising phosphatidylserine and coffee fruit extract.

### Phosphatidylserine

In one embodiment, the method comprises administering a dietary supplement composition comprising phosphatidylserine. In one embodiment, phosphatidylserine may be sourced from a plant. In particular, phosphatidylserine may be sourced from soybeans. This is a preferred alternative to animal sources to render the dietary supplement composition vegetarian or vegan.

There is no recommended daily intake for phosphatidylserine, however, the inventors have found suitable quantities for achieving the effects of improved brain function. In some embodiments, the composition of the invention may comprise phosphatidylserine in a unit dose from about 40 mg to about 400 mg. In some embodiments, the composition of the invention may comprise phosphatidylserine in a unit dose from about 90 mg to about 300 mg, or from about 100 mg to about 200 mg. In an embodiment, a dietary supplement composition may be administered to a target subject one or more times a day. In this case, a number of unit doses can be administered to achieve a desired daily intake amount of phosphatidylserine, for example from about 40 mg to about 400 mg or it can be administered in a single dose. In some embodiments, the composition of the invention may be administered in one or more unit doses to deliver phosphatidylserine in an amount from about 90 mg to 300 mg, or from about 100 mg to about 200 mg.

### Coffee Fruit Extract

In one embodiment, the method comprises administering a dietary supplement composition comprising coffee fruit extract.

In an embodiment, a source of coffee fruit extract is from the whole fruit of the Coffea arabica plant. The extract may optionally be treated to remove caffeine.

There is no recommended daily intact for coffee fruit extract, however, the inventors have found suitable quantities for achieving the effects of improved brain function. In some embodiments, the composition of the invention may comprise coffee fruit extract in a unit dose from about 80 mg to about 300 mg per serving. In some embodiments, the composition of the invention may comprise coffee fruit extract in a unit dose from about 100 mg to about 240 mg.

In an embodiment, a dietary supplement composition may be administered to a target subject one or more times a day. In this case, a number of unit doses can be provided to achieve a desired daily intake of coffee fruit extract, for example from about 80 mg to about 300 mg or it can be administered in a single unit dose. In some embodiments, the composition of the invention may be administered in one or more unit doses to deliver coffee fruit extract in an amount from about 100 mg to about 240 mg.

Together the phosphatidylserine and coffee fruit extract support neuronal health and increase BDNF in a target subject. Though not wishing to be bound by theory, it is believed that phosphatidylserine and coffee fruit extract interact synergistically with one another to provide unexpected benefits when compared to the effect achieved by each of these ingredients taken alone. Improved brain function can be demonstrated in a target subject by an improvement in performance in accuracy, concentration, focus, learning and memory. Examples of how this can be verified is through the performance of tasks, for example by assessments, puzzles, games and mental exercises which can be monitored by recording the results and the time taken to complete each task. This can be tracked and monitored to determine the improvement in brain function from before and during the administration of the dietary supplement composition.

The dietary supplement composition of the present invention may comprise additional ingredients. Non-limiting examples of such ingredients can include but are not limited to further active ingredients, excipients, flow agents, processing aids, solvents, carriers, antioxidants, lubricants, colorants, flavourings and the like and will be apparent to those skilled in the art in view of the disclosure herein.

The nutritional composition may optionally include, but is not limited to, one or more of the following vitamins or derivations thereof: vitamin B6, vitamin B12 and/or folate. The source of vitamin B6 may be pyridoxine, pyridoxal, pyridoxamine or pyridoxine hydrochloride and be in an amount of about 1 mg to 4 mg, more preferably about 1.5 mg to about 2.5 mg per unit dose. The source of vitamin B12 may be cobalamin, methylcobalamin, deoxyadenosylcobalamin, cyanocobalamin, hydroxocobalamin or adenosylcobalamin and be in an amount of about 1 μg to about 11 μg, preferably about 2 μg to about 8 μg and more preferably about 2.4 μg to about 4.8 μg per unit dose. The source of folate may be from folic acid, folacin or pteroylglutamic acid. Preferably, the folate is provided by folic acid in an amount of about 0.2 mg to about 1.0 mg, preferably about 0.4 mg to about 0.8 mg per unit dose. When the combination of vitamin B6, vitamin B12 and folate are included together in the dietary supplement composition the improved brain function can be further demonstrated by improved reasoning capability of the target subject. Again, this can be proven through the performance of tasks.

In other embodiments, excipients that can be used include but are not limited to rice bran, microcrystalline cellulose, gelatin, pectin, hydroxypropyl methylcellulose, hydroxypropyl cellulose, polyvinylpyrrolidone, calcium phosphate, dicalcium phosphate, sucrose, starch, maltodextrin, inulin, agar, acacia, dextrose and stearic acid. If rice bran is used in the dietary supplement composition, then it is present in an amount of about 4 mg to about 25 mg per unit dose.

In other embodiments, the comprises at least one flow agent. Flow agents can include but are not limited to silicon dioxide, talc, magnesium silicate, calcium silicate, calcium phosphate and starch.

In an embodiment, the total amount of flow agents may be from about 4 mg to about 15 mg per unit dose.

In other embodiments, the dietary supplement composition comprises other active ingredients. For example, one or more vitamins and/or minerals may also be added into the nutritional composition in amounts sufficient to supply at least some of the daily nutritional requirements of a subject. It is to be understood by one of ordinary skill in the art that vitamin and mineral requirements will vary, for example, based on the age of the target individual. Thus, the embodiments are not intended to limit the nutritional composition to a particular age group but, rather, to provide a range of acceptable vitamin and mineral components.

In other embodiments, the dietary supplement composition comprises at least one lubricant. Lubricants can include but are not limited to magnesium stearate, calcium stearate, glycerine monostearate, glyceryl behenate, hydrogenated vegetable oil, mineral oil, medium chain triglycerides, sodium lauryl sulfate, stearic acid and talc.

In other embodiments, the dietary supplement composition comprises at least one colorant. Colorant can include coloring agents derived either from natural or synthetically prepared sources. Any coloring agents approved by the Food and Drug Administration for use in human food and/or drugs are contemplated by the present invention.

In other embodiments, the dietary supplement composition comprises flavoring. Flavoring can include but is not limited to ethyl acetate, ethyl vanillin, fumaric acid, leucine, malic acid, maltol, menthol, vanillin, acesulfame potassium, citric acid, fructose, ethylcellulose, tartaric acid, trehalose and xylitol.

The compositions according to the present invention may be formulated in a variety of forms for administration and comprise an effective amount of phosphatidylserine and coffee fruit extract, by which is meant an amount sufficient to provide health benefits to a target subject. In some embodiments, the effective amounts of phosphatidylserine and coffee fruit extract are sufficient to improve brain function.

The compositions presently disclosed may be administered orally. Any dosage forms, and combinations thereof, are contemplated by the present invention. Examples of dosage forms suitable for oral delivery include, without limitation, chewable tablets, elixirs, liquids, solutions, suspensions, emulsions, capsules, soft capsules, hard capsules, tablet, powder, gummy, melt in the mouth, caplets, lozenges, confections and combinations thereof. The capsules may be hard capsules, e.g. two-piece capsules, or soft capsules, e.g. softgels. In at least one embodiment, the capsule is a hard shell.

The composition may be encapsulated in a capsule made from a non-mammalian source such as hydroxypropyl methyl cellulose, carrageenan and pectin which renders the dietary supplement composition vegetarian or vegan. Such hardshell capsules can be prepared, by way of example, without limitation, by dispersing the formulation in an appropriate vehicle, such as rice bran, and then encapsulating in a film to form a capsule in any machinery known to those in the industry of manufacturing.

### EXAMPLES

Examples are provided to illustrate some embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with the examples, be considered to be exemplary only, with the scope and spirit of the disclosure being indicated by the claims which follow the examples.

### Examples 1-4

Dietary supplement compositions of the disclosure may comprise the ingredients in the following amounts to form a unit dose.

TABLE 1

| | Exam- | Exam- | Exam- | Exam- |
|---|---|---|---|---|
| Ingredients | ple 1 | ple 2 | ple 3 | ple 4 |
| Phosphatidylserine (mg) | 40 | 100 | 200 | 400 |
| Coffee fruit extract (mg) | 80 | 100 | 240 | 300 |
| Rice bran (mg) | 7 | 18 | 21 | 24 |
| microcrystalline cellulose (mg) | 100 | 130 | 120 | 150 |
| silicon dioxide (mg) | 6 | 7 | 8 | 8 |
| Pyridoxine hydrochloride | | 1 | | 4 |
| (vitamin B6) (mg) | | | | |
| Cyanocobalamin (vitamin B12) | | 3 | | 8 |
| (mcg) | | | | |
| Folic acid (from folate) (mg) | | 0.4 | | 0.6 |
| Magnesium stearate (mg) | | 3 | | 4 |

The dietary supplement composition of Example 1 may be suitable for use in a dosage regime where three unit doses can be taken in a day to provide 120 mg of phosphatidylserine and 240 mg of coffee fruit extract. The dietary supplement composition of Example 2 may be suitable for use in a dosage regimen where one or more unit doses are administered per day.

The dietary supplement compositions of Examples 3 and 4 may be suitable for use in a dosage regimen where one unit dose is administered per day.

### Example 5

One exemplary composition comprising phosphatidylserine and coffee fruit extract according to the present invention was prepared according to the formulation provided in Table 2. Phosphatidylserine was purchased from Enzymotec under the brand name Sharp-PS® which contains further ingredients. Coffee fruit extract was purchased from Futureceuticals.

TABLE 2

| Ingredient | Amount per unit dose | |
|---|---|---|
| Phosphatidylserine | 120 | mg |
| Coffee fruit extract | 120 | mg |
| Rice bran | 8 | mg |
| microcrystalline cellulose | 132.3 | mg |
| silicon dioxide | 8 | mg |

The composition was encapsulated in a film comprising: hydroxypropyl methylcellulose sold by Suheung to form a hardshell capsule. The resulting unit dose had a total fill of 468.3 mg with a shell weighing 95 mg. Such a unit dose may be used in a dosage regimen where one unit dose is administered per day.

### Example 6

Another exemplary composition comprising phosphatidylserine and coffee fruit extract according to the present invention is provided in Table 3. Phosphatidylserine was purchased from Enzymotec under the brand name Sharp-PS® which contains further ingredients. Coffee fruit extract was purchased from Futureceuticals.

TABLE 3

| Ingredients | Amount per unit dose | |
|---|---|---|
| Phosphatidylserine | 120 | mg |
| Coffee fruit extract | 240 | mg |
| Pyridoxine hydrochloride (vitamin B6) | 2.55 | mg |
| Cyanocobalamin (vitamin B12) | 0.01139 | mg |
| Folic acid (from folate) | 0.79928 | mg |
| Rice bran | 21 | mg |
| microcrystalline cellulose | 118 | mg |
| silicon dioxide | 8 | mg |
| Magnesium stearate | 4 | mg |

The composition was encapsulated in a film comprising: hydroxypropyl methylcellulose sold by Suheung to form a hardshell capsule. The resulting unit dose soft gel had a total fill of 595.03 mg with a shell weighing 118 mg. Such a unit dose may be used in a dosage regimen where one unit dose is administered per day.

### Example 7

Another exemplary composition comprising phosphatidylserine and coffee fruit extract according to the present invention is provided in Table 4. Phosphatidylserine was purchased from Enzymotec under the brand name Sharp-PS® which contains other ingredients. Coffee fruit extract was purchased from Futureceuticals.

TABLE 4

| Ingredients | Amount per unit dose | |
|---|---|---|
| Phosphatidylserine | 100 | mg |
| Coffee fruit extract | 200 | mg |
| Pyridoxine hydrochloride (vitamin B6) | 1.7 | mg |
| Cyanocobalamin (vitamin B12) | 0.0024 | mg |
| Folic acid (from folate) | 0.4 | mg |
| Rice bran | 21 | mg |
| microcrystalline cellulose | 118 | mg |
| silicon dioxide | 8 | mg |
| Magnesium stearate | 4 | mg |

The composition was encapsulated in a film comprising: hydroxypropyl methylcellulose sold by Suheung to form a hardshell capsule. Such a unit dose may be used in a dosage regimen where one unit dose is administered per day.

The compositions made according to the invention provide benefits to a target subject when administered. These benefits include improved brain function demonstrated by improved focus, memory, learning, accuracy and concentration.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references. Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. For example, while methods for the production of a commercially sterile liquid nutritional supplement made according to those methods have been exemplified, other uses are contemplated. Therefore, the spirit and scope of the appended claims should not be limited to the description of the versions contained therein.

1. A method of improving brain function in a target subject, the method comprising:
administering a dietary supplement composition comprising:
phosphatidylserine; and
coffee fruit extract.

2. The method according to claim 1, wherein the phosphatidylserine is in an amount of about 75 mg to about 400 mg, preferably about 90 mg to about 300 mg and more preferably about 100 to about 200 mg.

3. The method according to claim 1, wherein the coffee fruit extract is in an amount of about 80 mg to 300 mg and more preferably about 100 mg to 240 mg.

4. The method according to claim 1, wherein the dietary supplement composition further comprises one or more vitamins.

5. The method according to claim 4, wherein the vitamins comprise at least one of vitamin B6 and vitamin B12.

6. The method according to claim 5, wherein the vitamin B6 is provided by one or more of pyridoxine, pyridoxal, pyridoxamine and pyridoxine hydrochloride.

7. The method according to claim 6, wherein the vitamin B6 is in an amount of about 1 mg to 4 mg, more preferably about 1.5 mg to about 2.5 mg.

8. The method according to claim 5, wherein the vitamin B12 is provided by one or more of cobalamin, methylcobalamin, deoxyadenosylcobalamin, cyanocobalamin, hydroxocobalamin and adenosylcobalamin.

9. The method according to claim 8, wherein the vitamin B12 is in an amount of about 1 μg to about 11 μg, preferably about 2 μg to about 8 μg and more preferably about 2.4 μg to about 4.8 μg.

10. The method according to claim 5, wherein the dietary supplement composition further comprises folate.

11. The method according to claim 10, wherein the folate is provided by at least one of folic acid, folacin and pteroylglutamic acid.

12. The method according to claim 11, wherein the folic acid is present in an amount of about 0.2 to about 1.0 mg, preferably about 0.4 mg to about 0.8 mg.

13. The method according to claim 1, wherein the dietary supplement composition further comprises one or more ingredients selected from the group consisting of solvents, carriers, flow agents, antioxidants, excipients, lubricants, processing aids, colorants, water, and combinations thereof.

14. The method according to claim 1, wherein the dietary supplement composition is in a form selected from the group consisting of a solution, a capsule, a tablet, a gummy, a chewable, a melt in the mouth form, a liquid or a caplet.

15. The method according to claim 1, wherein the target subject is an adult human.

16. The method according to claim 1, wherein the coffee fruit extract has been decaffeinated.

17. A dietary supplement composition comprising:
phosphatidylserine; and
coffee fruit extract.

18. The dietary supplement composition according to claim 17, wherein the phosphatidylserine is in an amount of about 75 mg to about 150 mg, preferably about 90 mg to about 130 mg and more preferably about 100 to about 200 mg.

19. The dietary supplement composition according to claim 17, wherein the coffee fruit extract is in an amount of about 80 mg to 300 mg and more preferably about 100 mg to 240 mg.

20. The dietary supplement composition according to claim 17, wherein the supplement further comprises one or more vitamins.

21. The dietary supplement composition according to claim 20, wherein the vitamins comprise at least one of vitamin B6 and vitamin B12.

22. The dietary supplement composition according to claim 21, wherein the vitamin B6 is provided by one or more of pyridoxine, pyridoxal, pyridoxamine and pyridoxine hydrochloride.

23. The dietary supplement composition according to claim 22, wherein the vitamin B6 is in an amount of about 1 mg to 4 mg, more preferably about 1.5 mg to about 2.5 mg.

24. The dietary supplement composition according to claim 21, wherein the vitamin B12 is provided by one or more of cobalamin, methylcobalamin, deoxyadenosylcobalamin, cyanocobalamin, hydroxocobalamin and adenosylcobalamin.

25. The dietary supplement composition according to claim 24, wherein the vitamin B12 is in an amount of about 1 μg to about 11 μg, preferably about 2 μg to about 8 μg and more preferably about 2.4 μg to about 4.8 μg.

26. The dietary supplement composition according to claim 21 further comprising folate.

27. The dietary supplement composition according to claim 26, wherein the folate is provided by at least one of folic acid, folacin and pteroylglutamic acid.

28. The dietary supplement composition according to claim 27, wherein the folic acid is present in an amount of about 0.2 to about 1.0 mg, preferably about 0.4 mg to about 0.8 mg.

29. The dietary supplement composition according to claim 17 further comprising one or more ingredients selected from the group consisting of solvents, carriers, flow agents, antioxidants, excipients, lubricants, processing aids, colorants, water, and combinations thereof.

30. The dietary supplement composition according to claim 17, wherein the supplement is in a form selected from the group consisting of a solution, a capsule, a tablet, a gummy, a chewable, a melt in the mouth, a liquid or a caplet.

31. The dietary supplement composition according to claim 17, wherein the coffee fruit extract has been decaffeinated.

32. A dosage regimen for administering a dietary supplement composition to a target subject, the dietary supplement composition comprising:
phosphatidylserine; and
coffee fruit extract,
wherein the dosage regimen comprises a daily dosage amount of the phosphatidylserine and the coffee fruit extract, optionally delivered in one or more unit doses.
